(19) United States
(12) Patent Application Publication
(10) Pub. No.: US 2025/0213606 A1
(43) Pub. Date: Jul. 3, 2025

(54) MEDICINAL AGENT (EMBODIMENTS) AND DEVICE FOR USING SAME

(71) Applicant: Vasilii Aleksandrovich PETROV, Saint-Petersburg, pos. Levashovo (RU)

(72) Inventors: Vasilii Aleksandrovich PETROV, Saint-Petersburg, pos. Levashovo (RU); Andrey Olegovich IVANOV, Saint-Petersburg (RU); Anatoliy Valerievich KINDZERSKIY, Saint-Petersburg (RU); Ivan Viktorovich MAYOROV, Saint-Petersburg (RU); Iaroslav Vasilievich KUDANOV, Saint-Petersburg (RU)

(21) Appl. No.: 18/996,680

(22) PCT Filed: Mar. 17, 2023
(86) PCT No.: PCT/RU2023/050052

§ 371 Date: Jan. 17, 2025

(30) Foreign Application Priority Data
Jul. 19, 2022 (RU) 2022119725

(51) Int. Cl. A61K 33/00; A61M 16/12

(52) U.S. Cl. CPC A61K 33/00; A61M 16/12; A61M 2202/0208; A61M 2202/025; A61M 2202/0291

(57) ABSTRACT

The inventions are intended for stopping or slowing the progression of the acute phase of ischemic stroke, coronary failure and respiratory failure, and for saving injured and wounded persons suffering from heavy blood loss. A medicinal agent according to a first embodiment contains from 40 to 50 vol % helium, from 25 to 35 vol % argon, from 0.2 to 5 vol % xenon, from 2 to 10 vol % krypton, and not less than 21 vol % oxygen, to a total of 100 vol %, and the temperature of the gas mixture is 30-70° C. A medicinal agent according to a second embodiment contains from 21 to 60 vol % oxygen, from 0.2 to 35 vol % xenon, from 0.2 to 35 vol % krypton, or from 21 to 60 vol % oxygen, xenon in a concentration range of from 0.2 to 35 vol %, from 0.2 to 35 vol % krypton, and the remainder argon in an amount of not less 25 vol %, and the temperature of the gas is 15-70° C. The claimed device is characterized by a mask 1 connected to a breathing bag 7 and to a canister 12 containing an argon-xenon-krypton gas mixture, with a three-way valve for alternately supplying gases to the breathing bag 7, the device being further equipped with a canister 9 containing a helium-oxygen-argon-xenon-krypton gas mixture and having a valve mounted thereon with a manometer that is connected to the mask to permit a supply of the gas mixture directly into the mask 1, and a chemical or electric heater 11 with a connecting heat exchange line.

### TECHNICAL AREA

The invention relates to the field of medicine, in particular, to medical and sanitary care, emergency medical care, including emergency specialized medical care, military field therapy, disaster medicine.

The medicinal product is intended for relief and inhibition of the development of the acute phase of ischemic stroke, coronary and respiratory insufficiency, rescue of injured and injured with large blood loss, concomitant exogenous and endogenous hypothermia, for effective use, mainly at home or in the field, before the arrival of an ambulance team, as well as at the stage of delivery to medical institution and in a medical institution.

### RELATED ART

According to the related art medicines and the procedure for their use are known to maintain the life of a person with severe blood loss, acute cerebral and myocardial ischemia, acute respiratory failure, and hypothermia.

The procedure for providing medical care for the development of strokes and heart attacks at the pre-medical stage is described in the guidelines for a wide range of the population. These instructions suggest the simplest measures that will not be able to harm the patient, but in some cases, measures with the use of medical procedures and medicines (see, for example, https://medbe.ru/materials/ostrve-zabolevaniya/neotlozhnava-pomoshch-pri-ostrykli-narusheniyakh-mozgovogo-krovoobrashchenia/ on the website www.MedBe.ru and https://doktor-ok.com, “8 main rules of first aid in the case of stroke”, etc.).

In this case, the standard procedure for pre-medical care involves:
ensuring the patency of the upper respiratory tract;
inhalation of oxygen through a nasal catheter;
relief of psychomotor agitation by intramuscular administration of the 1-2 ml of 3% phenazepam solution;
in the case of the increased blood pressure: intramuscularly, 2-4 ml of 2% of papaverine solution and 2-4 ml of 1% dibazole solution;
in the case of arterial hypotension: 2-4 ml of cordiamine or 2-4 ml of 10% of sulfocamphocaine solution.

Primary actions in the case of wounds consist of a set of measures, including the prompt prevention of blood loss by applying bandages and tourniquets, the administration of anti-shock medicines.

In general, such a scheme is aimed at preventing hypoxia of the brain and heart muscle, controlling hypovolemia, relieving pain shock and ensuring the preservation of vital functions until the arrival of an ambulance.

It is obvious that the set of proposed measures is difficult to implement in the domestic conditions of pre-medical care in the absence of specialists.

In a hospital setting, one of the main directions of specialized medical care in the acute phase of ischemic stroke and coronary insufficiency is also the provision of antihypoxic and thrombolytic therapy, the introduction of antiplatelet agents and neuroprotectors. The most important task is to prevent prolonged ischemia of the penumbra zone and the expansion of the infarction zone.

Medicinal methods include the administration of medicines with antihypoxic, neuroprotective and nootropic effects (antihypoxants). Thus, there is a method of protecting nerve cells in acute stroke by injecting the drug noopept (RF patent No. 2330680, IPC A61K 38/05, A61K 31/401, A61K 9/08, A61K 9/19, A61J 3/00, dated 10 Aug. 2008). The use of the Noopept may lead to a significant decrease in the area of brain damage in experimental ischemia.

Another class of antihypoxants includes, for example, the medicine Hypoxen (sodium polydihydroxyphenylenethiosulfonate), taken orally and regulating cell metabolism (RF Patent No. 2105000, C07C 381/02, C08G 61/10, A61K 31/05, publ. Feb. 20, 1998; U.S. Pat. No. 6,117,970, IPC A61RZ/00; A61R43/00; C07C3 81/02; C08G61/10, dated 12 Sep. 2000). Hypoxene restores the process of macroerg generation, disrupted or interrupted by certain pathological processes. It is used to treat ischemic damage to the central nervous system, chronic fatigue syndrome, intoxication with hypoxic poisons and other chronic and acute hypoxic conditions.

The disadvantages of existing measures to prevent blood loss at the pre-medical stage are the need for the participation of medical personnel, low effectiveness in deep or widespread lesions, inability to use for preventive purposes, side effects, contraindications, high cost of medicines.

Other agents with antihypoxic effects include the use of gas mixtures with a high oxygen content.

Thus, an effective method of hyperbaric oxygen therapy is known, consisting in the treatment of oxygen under high pressure (see, for example, Petrovsky B. V., Efuni S. N. Fundamentals of hyperbaric oxygenation.—M.: Medicine, 1976.—344 p.; Artru F., Charcornac R., Deleuze R. Hyperbaric oxygenation for severe head injuries: Preliminary results of a controlled study.—Eur Neurol., 1976, —V. 14—p. 310-318; Murthy T. Role of hyperoxia and hyperbaric oxygen in severe head injury: A review.—Indian Journal of Neurotrauma, 2006.—Vol 3.— [symbol] 2—p. 77-80). The therapeutic effect of hyperbaric oxygen therapy is based on a significant increase in oxygen tension in the body's liquid media (blood, lymph, tissue fluid), which allows for rapid oxygen delivery to tissues suffering from hypoxia and promotes the restoration of cellular respiration. However, the key disadvantage of using oxygen under high pressure is possible complications, mainly in the form of an increase in intracranial pressure.

The another effective way to stop the acute development of ischemic stroke, heart attack, acute respiratory failure and hypovolemia is the use of oxygen antihypoxic therapy using a ventilator (see “Features of oxygen transport in the acute period of ischemic stroke/K. V.Lukashev et al.//V.A. Netovsky Research Institute of General Resuscitation of the Russian Academy of Medical Sciences, electronic resource: cyberleninka.ru, 2010).

A known method of oxygen therapy for stroke, heart attack, coronary heart disease and other diseases in their acute phase, accompanied by hypoxia, in the provision of pre-medical care (including self-help), implemented using portable oxygen cylinders, breathing of which is aimed at reducing hypoxic phenomena (see, for example, electronic resource: medprep.info/oxygen; or an electronic resource: med.ru/the use of oxygen therapy for heart diseases, etc.).

One cylinder can provide respiration with an oxygen-enriched mixture for several minutes, for example:
Spare Air 0.28 liter, 20 MPa, air capacity under normal conditions (not stated)
56 liter, diameter 5.71 cm, length 22.23 cm, weight 0.687 kg, refueled (about 6 minutes of application);
Spare Air 0.42 liter, 20 MPa, air capacity at normal conditions
82 liter, diameter 5.71 cm, length 34 cm, weight 0.985 kg, refueled (about 10 minutes of use) and others.

A method of using an oxygen canister “Air-Active” in the form of inhalations was described in order to eliminate oxygen starvation (hypoxia), normalize metabolism in the human body, increase mental and physical performance and endurance, as well as to prepare oxygen cocktails in everyday conditions. The oxygen canister “AirActive” contains a gas mixture of the composition Ag—25%, 02—75% (manufacturer OOO “Tyumen Aerosols”).

The specified mixture of oxygen with argon is more effective for the purpose of preventing hypoxia of organs, but the argon content in this mixture is at the level of 25% by volume, the absence of other effective components in it, for example, xenon, and a small supply of the gas mixture, which does not allow the patient to breathe during the entire period of time, which in the most cases required for the arrival of an ambulance limit the effectiveness of this method for the purpose of relieving acute ischemic attacks in the provision of pre-medical care.

In case of aggravation of the situation by exposure to the patient or the wounded to factors of the cooling climate and endogenous factors associated, for example, with blood loss, the risk of formation of terminal and irreversible conditions increases sharply, requiring additional measures to maintain temperature homeostasis. However, the range of such activities is severely limited in home and field conditions, and, as a rule, is limited only to the use of additional equipment, which is often ineffective due to blood loss, forced immobilization of the sick or injured, and the intake of cold air into the body.

Therefore, in these conditions, the resolution of the situation is only the urgent delivery of the wounded to an inpatient facility, in which it is possible to carry out the required amount of intensive care, including warming measures.

However, most often, the emergency delivery to a medical facility is difficult. In such situations, the development of medicines and devices for stabilizing and maintaining the pre-terminal condition of the patient or injured and preventing combined hypothermia for a long time becomes particularly relevant.

In inpatient conditions, resuscitation measures are used in order to prevent or withdraw from a terminal condition, including infusion therapy aimed at eliminating hypovolemia.

Hemodynamic blood substitutes are also widely used to correct hypovolemia: dextran preparations (Rheopolyglucin, Polyglucin), gelatin solutions (Gelatinol), hydroxyethyl starch (Refortan, Stabizol, Infucol), saline solutions (Saline solution, Ringer lactate, Lactosol), sugar solutions (Glucose, Glucosteril). Of the blood products, erythrocyte mass, freshly frozen plasma, and albumin are more often used. In the absence of an increase in blood pressure, despite adequate infusion therapy for 1 hour, drugs such as adrenaline, norepinephrine, dopamine and other vasoconstrictor medicines are additionally administered (after stopping bleeding) (see, for example, at: http://medbe.ru/materials/obshchie-voprosy-vpkh/pervava-meditsinskaya-pomoshch-na-pole-boya-i-v-ochage-massovykh-poter).

In the treatment of hemorrhagic shock, medicines that improve the rheological properties of blood are used therefore: Heparin, Curantil, Trental, as well as corticosteroids. After removing the patient from hemorrhagic shock and eliminating the immediate threat to life, correction of violations of individual links of homeostasis (acid-base composition, hemostasis, and so on) is performed.

However, only a limited set of emergency therapeutic measures and medications used is possible at home or in the field.

Currently, there are no effective medicines and methods to provide relief and inhibition of the development of the acute phase of ischemic stroke, coronary and respiratory failure, rescue of the injured and injured with large blood loss and concomitant exogenous and endogenous hypothermia in home or field conditions.

It is known to use a mixture of gases of the composition: Xenon 1-10% by volume, Argon 30-35% by volume, Oxygen 60-65% by volume, for the relief of acute ischemic attacks according to patent RU No. 2748126 dated 19 May 2021, “A method for emergency relief of acute ischemic attacks with impaired cerebral or coronary circulation”, in which the patient is given an inhalation drug in the form of a mixture of gases. The disadvantages of the medicine include its inapplicability under cold conditions due to the possible condensation of xenon, which is associated with its critical temperature of 289.74 K (16.59° C.).

Medicines used for high blood loss, in the form of medical gaseous oxygen or a mixture of oxygen and nitrogen with a high oxygen content of more than 60% are known as well. The use of these gas mixtures helps to increase blood oxygen saturation, but does not provide warming and oxygen protection for the most important organs. In addition, to provide long-term therapy during the evacuation of the wounded, a large volume of oxygen or an oxygen-containing mixture is required, which cannot be provided in the field. The toxic effect of the high oxygen content in the mixture is also negative.

A mixture of gases “Heliox” is known, which makes it possible to optimize the temperature regime of the body. The heated Heliox mixture evenly warms the parenchyma of the chest organs, quickly relieves hypothermia of the body. These effects are associated with high thermal conductivity and the highest diffusion capacity of helium (see L. V. Shogenova, “The effects of using HELIOX as a working gas during inhalation of (32-agonists using a nebulizer in patients with exacerbation of asthma”. Federal State University “Research Institute of Pulmonology”—FMBA of Russia, Moscow, “Effective pharmacotherapy. Pulmonology and otorhinolaryngology”—No. 2.—2010.). The mixture can also be used to treat acute respiratory insufficiency (The use of thermal Heliox (t-He/02) in the treatment of patients with respiratory insufficiency (syndrome of respiratory disorders)/Clinical recommendations of the Russian Federation 2018-2020 (Russia)/https://medelement.com). However, this mixture of gases, when using the claimed technical means, affects the body only in the short term, and its composition does not sufficiently ensure an antihypoxic effect and long-term maintenance of the viability of the wounded with massive blood loss, and for a long time to stop an attack of acute respiratory failure.

Therefore, “Method of exposure of gas mixtures to the body” are known, according to the RF patent No. 2232013, IPC A61K 31/02, A61M 16/00, A61R 43/00, dated 10 Jul. 2004” which involves exposure to the body with a gas mixture containing oxygen and at least one diluent gas, while therapeutic. The action is carried out by changing the conditions and mode of exposure with periodic replacement of one gas mixture with another, as well as by changing at least one of the physical characteristics of the injected mixture and/or its parameters, while oxygen is maintained within 12-85% by volume. This invention contributes to the activation of reductive-oxidative and energy processes in the body, increasing its specific adaptation to environmental changes.

However, the disadvantages of using a gas mixture include the insufficiently effective effect of the drug in maintaining the life of patients with severe blood loss, acute cerebral and myocardial ischemia, acute respiratory failure and hypothermia for use at the first emergency stage in the field, at home and in an ambulance.

There is a known method of rehabilitation of a person with hypothermia according to the RF patent No. 2275211, IPC A61M 16/00, dated 27 Apr. 2006, which provides rehabilitation of a person with exogenous hypothermia by inhalation warming and oxygenation of the body with a heated and moistened artificial respiratory gas mixture, characterized in that inhalation warming and oxygen supply of the body is carried out with an artificial respiratory gas mixture containing: oxygen 49-51% by volume, the rest is helium, at a mixture temperature of 40-44° C.

The disadvantage of this gas mixture and the method of its application is the absence of additional (other than oxygen therapy) antihypoxic effects of such an artificial respiratory mixture necessary to maintain the life of patients with an ischemic attack, acute respiratory failure or with severe blood loss.

The closest in technical essence to the claimed invention is a drug in the form of a mixture of gases proposed in the invention “A method for long-term maintenance of human viability in the field with wounds with severe blood loss and a device for its implementation” according to the RF patent No. 2684748, IPC A61M 16/12, dated 12 Apr. 2019, adopted as a prototype, which ensures the long-term maintenance of the vitality of the wounded while breathing the mixture. The gas mixture contains up to 1-35% by volume of xenon, 30-35% by volume of argon, at least 21% by volume of oxygen and nitrogen—by the rest.

The disadvantage of this gas mixture for the purposes of the present invention is the absence of a set temperature of the medicine for a purposeful warming antihypothermic effect on the patient, injured and wounded, and the absence of krypton gas in the gas mixture, which completes the analgesic effect of the proposed respiratory gas mixture and ensures the use of the drug at low temperatures.

The closest in technical essence to the claimed device for supplying medicinal gas is the device described in the invention under the RF patent No. 2684748, IPC A61M 16/12, dated 12 Apr. 2019, adopted as a prototype, including the front part—a mask, a breathing bag with an overpressure valve, a frame, a connecting tube, a replaceable regenerative cartridge, a mouthpiece located in the mask, a dispenser bag with a switchable check valve and a connecting line, a cylinder with an argon-xenon gas mixture of up to 0.5 liters with a pressure of up to 30 MPa, or two separate cylinders of a smaller capacity with argon and xenon in the assembly and with a three-way valve for alternate gas supply, a mixture supply regulator, a connecting line for feeding the mixture into a dispenser bag, as well as a mixture pressure sensor in the cylinder, A wrist-mounted portable computer with heart rate (HR) and blood oxygen saturation sensors, an oxygen and xenon concentration sensor in a breathing bag.

However, this device does not contain a container and a gas path for storing and supplying the first warming respiratory mixture of gases, there is no sensor for monitoring the concentration of the argon-krypton-xenon mixture and, thus, cannot ensure sufficient effectiveness of the invention.

### SUMMARY OF INVENTION

The invention is aimed at developing therapeutic agents for maintaining the life of patients with severe blood loss, acute cerebral and myocardial ischemia, acute respiratory failure and hypothermia for use at the initial preclinical stage of the development of events, the stage of first emergency care in the field, at home, in an ambulance, in a hospital at the stages of preparation for surgery, surgery, intensive care and rehabilitation and possible for use in low temperature conditions.

### Technical Task

The technical result of the implementation of the claimed invention is to reduce mortality and disability in wounds and injuries with severe blood loss, acute cerebral and myocardial ischemia, acute respiratory failure and, including hypothermia, for use at the stage of first aid in the field, at home and in an ambulance, as well as in medical institutions, the possibility of using the drug at low temperatures.

### Solving the Problem

A medicine for maintaining the life of a person with severe blood loss, acute cerebral and myocardial ischemia, acute respiratory failure, and hypothermia, according to Option 1, is a medical gas including oxygen, xenon and argon, and differs in that it contains helium in the concentration range from 40 to 50% by volume, argon in the concentration range from 25 to 35% vol., xenon in the concentration range from 0.2 to 5% vol., krypton in the concentration range from 2 to 10% by volume and oxygen—at least 21% by volume total up to 100% by volume in total, and the temperature of the gas mixture is 30-70° C.

A medicine for long-term maintenance of viability of the wounded with severe blood loss, acute cerebral and myocardial ischemia, acute respiratory failure, and hypothermia, is a medical gas including oxygen, xenon, argon, and nitrogen, characterized in that it contains oxygen in the concentration range from 21% by volume to 60% by volume, xenon in the concentration range from 0.2 to 35% by volume, krypton in the concentration range from 0.2 to 35% by volume, argon the rest in a concentration of at least 25% by volume, and the gas temperature is 15-70° C.

### Positive Results of the Invention

The use of the claimed medicines and devices for their use will provide a reserve of time to save lives, up to 6 hours or more, for evacuation from the scene of the event from home and field conditions to a medical institution.

In order to save a patient with severe blood loss, acute cerebral and myocardial ischemia, acute respiratory failure and hypothermia at the stage of first aid in the field, at home and in an ambulance, the present invention claims medicinal products—therapeutic gases, which are a mixture of gases with an increased content of inert gases and oxygen:

The claimed first respiratory mixture of gases has the following therapeutic effects:
quickly warms the body and, above all, vital organs (brain, heart, lungs, etc.) due to the receipt of a warm mixture with a high content of helium, which helps accelerate heat transfer to the deep tissues of the body;
provides bronchodilator effect due to the action of helium and argon;
increases the rate of oxygen transport in conditions of hypovolemia due to the specific effect of helium on reducing blood viscosity and transcapillary transport of gases;
reduces the phenomena of pain shock, provides an improvement in the psycho-emotional state of the wounded due to the psychotropic and analgesic effects of xenon or krypton;
facilitates the transfer of oxygen to the cells of vital organs due to the special (protective) effects of argon;
reduces oxygen demand and energy needs of cells, tissues and the body as a whole as a result of the hibernatic action of xenon, the synergistic effect of xenon or (and) krypton and argon on the energy systems of cells.

The claimed second respiratory mixture of gases has the following therapeutic effects:
reduces the phenomena of pain shock, provides an improvement in the psychoemotional state of the wounded due to the psychotropic, analgesic effects of xenon in sub-narcotic and higher concentrations, analgesic effects of krypton;
facilitates the transfer of oxygen to the cells of vital organs due to the special (protective) effects of argon;
reduces oxygen demand and energy requirements of cells, tissues and the body as a whole as a result of the hibernatic action of xenon, the synergistic effect of xenon, krypton and argon on the energy systems of cells;
reduces the likelihood of thrombosis and promotes the lysis of blood clots due to the anticoagulant properties of krypton in a certain concentration;
maintains the body's thermal balance due to the temperature of the mixture;
increases the volumetric rate of oxygen transport into capillaries alveoli due to an increase in the concentration of oxygen in the respiratory mixture, the accelerating effect of argon, krypton and xenon on transmembrane transport, and the broncholytic effects of argon.

In order to realize the properties of the claimed drug variants, which are respiratory mixtures of gases, and for long-term maintenance of the viability of patients with ischemic attacks and acute respiratory failure, injured and injured with severe blood loss and exogenous and endogenous hypothermia at home and in the field, methods and devices for their use are described.

The medicinal products claimed in the present invention are used as follows:
first, the patient or injured person is transferred to the breathing mode with a mixture of gases according to Option 1 in the composition, depending on the external air temperature and the patient's condition, containing helium in the concentration range from 40 to 50% by volume, argon in the concentration range from 25 to 35% by volume, xenon in the concentration range from 0.2 to 5% by volume, krypton in the concentration range from 2 to 10% by volume and oxygen—at least 21% by volume, up to 100% by volume in total, and the temperature of the gas mixture is 30-70° C.

The mixture is served for 5 to 30 minutes or more under the supervision of a doctor to relieve hypothermia, enrich blood and tissues of important organs with oxygen, relieve pain shock, relieve an attack of acute ischemia and respiratory failure. The conventional name of the gas mixture is “Hearoxxen”—krypto”.

Next, the patient is fed a second gas mixture according to Option 2, containing oxygen in the concentration range from 21% by volume to 60% by volume, xenon in the concentration range from 0.2 to 35% by volume, krypton in the concentration range from 0.2 to 35% by volume, argon the rest in a concentration of at least 25% by volume, and the gas temperature is 15-70° C.,

The mixture is fed continuously for 20 minutes and up to 6 hours or more to stabilize the viability of the patient before the arrival of an ambulance or (and) delivery to a medical institution. The conventional name of the gas mixture is “Agohhep”—krypto”.

The second mixture provides prevention of hypothermia, enrichment of blood and tissues of the most important organs with oxygen, relief of pain shock, long-term maintenance of viability in case of massive blood loss, long-term relief of attacks of acute ischemia and respiratory failure.

In the absence of signs of endogenous hypothermia and combined acute respiratory failure, it is possible to effectively use only the second medicine—a mixture of gases “Agohhep—krypto”.

A particularly important role is played by the mixture “Agohhep”—krypto” for use at home while waiting for the arrival of ambulance specialists. The use of the medicine “Agohhep”—krypto” for 20 to 40 minutes from a compressed gas cylinder will most likely save lives and reduce the consequences of ischemic attacks and injuries with blood loss and wait for ambulance specialists.

As a result of exposure to the body of the claimed medicinal products—respiratory mixtures of gases, the threshold for the development of irreversible damage to cells and tissues of vital organs is “pushed back”, the viability of the patient and the wounded is prolonged, the necessary time is provided for his transportation to a medical institution to provide qualified assistance or delivery of blood substitutes.

The patient himself, the patient's assistant, a professional medical worker can apply the medicine (“Hearoxxen”—krypto” and (or) “Aroxxen”—krypto”) in the described way.

For the use of a medicinal product in conditions of being in a populated area, the availability of communication facilities and ambulance services, devices for storing and supplying medicine should ensure the supply of medicine for 20 to 40 minutes while waiting for the arrival of an ambulance. Such devices for self-use are high-pressure gas cylinders with a gear valve, a gas tube, a face mask, or a mouthpiece. The devices are easy to maintain, store and use, and do not require special medical training.

For the use of the medicine in areas of accidents and catastrophes remote from habitats or in the field, a device is needed that allows in the field to create and maintain a respiratory mixture of gases with a given concentration of components and at a given temperature for a long time. At the same time, it is important that the device is compact, easy to use, and has a small mass. The continuous operation time of the device should be at least 3-4 hours or more when breathing at rest.

### BRIEF DESCRIPTION OF DRAWINGS

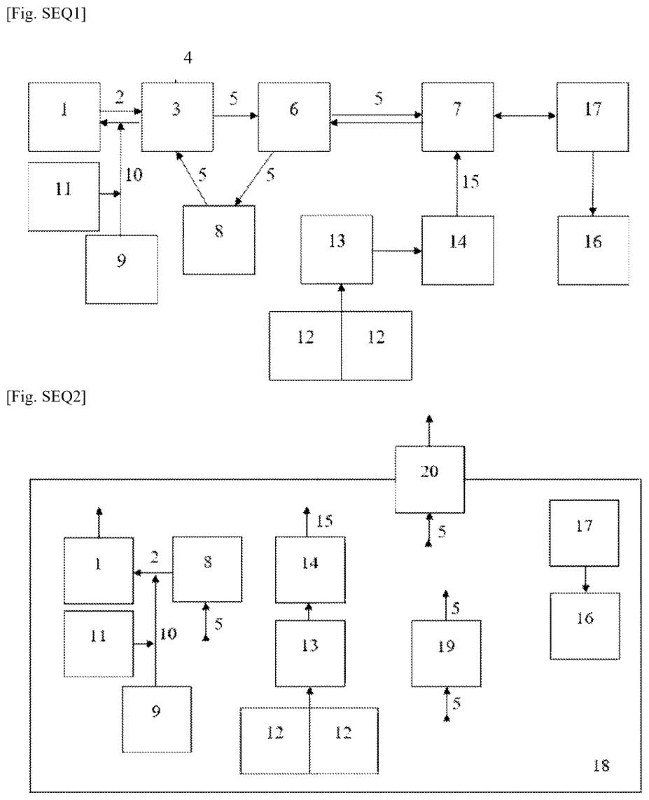

### FIG. 1

FIG. 1 The design of the device for the creation and use of the claimed medicines is shown in FIG. 1.

The device contains a mask 1 with a sub-mask and a mouthpiece, a connecting node 2 made in the form of a connecting tube with three outlets, a switchable block of a check valve 3, an exhaling outward line 4, connecting lines 5, a regenerative cartridge 6 (for example the one of RP-4P type with a starting briquette), a breathing bag 7 with an overpressure valve, a dispenser bag 8 with a check valve (may be the one of the Ambu type), a cylinder 9 of small volume (for example from 0.2 to 1 liter) with high pressure (for example, up to 30 MPa), with a valve and a pressure gauge mounted on it, filled with a helium-oxygen-argon-xenon-krypton mixture of gases, a heat exchange connecting line 10 made in the form of a heat exchanger, a chemical or electric heating pad with a battery 11, as well as a cylinder 12 with argon-xenon-krypton gas mixture of small volume (for example, from 0.2 to 1 liter), with high pressure (for example, up to 30 MPa), or several separate cylinders of smaller capacity with argon, xenon and krypton, or a mixture of xenon+krypton, assembled, equipped with valves and pressure gauges each. An assembly unit 13 with a three-way valve for supplying the gas mixture through the regulator 14 and a connecting line 15 to the breathing bag 7 is connected to the cylinder 12 (or to the cylinders in the assembly).

The Mask 1, connecting node 2, check valve block 3, connecting lines 5 and heat exchange connecting line 10, breathing bag 7, the dispenser bag 8 with a check valve are equipped with thermal insulation (not shown in the drawing).

In addition, in order to monitor and control the condition of the patient or the wounded, the device is equipped with a wrist laptop computer 16 with a sensor for heart rate (HR), blood oxygen saturation and temperature of the wounded, and a sensor 17 for the concentration of oxygen and a mixture of argon, xenon, krypton in the breathing bag 7. The device is placed in a packing bag (not shown in the drawing) and is equipped with a key, a stopper, and other accessories.

The device works as follows.

After the device is assembled, it is necessary to initiate a chemical or electric heating pad 11 in order to heat the heat exchange connecting line 10 and the breathing gas mixture in it to a temperature of 30-70° C., to open the valve for supplying the breathing gas mixture of the cylinder 9, to check its supply, to set the operating mode of the switchable check valve block 3 to line 4 after exhaling outwards; the mask 1 is put on the patient or injured person, the first mixture of gases is supplied for a given time (from 5 to 30 minutes or more) in order to relieve hypothermia. Before completing the gas supply, controlled by the clock and the pressure gauge readings on the cylinder 9, the regenerative cartridge 6 is initiated by means of using a starting briquette, while the breathing bag 7 is filled with oxygen from the regenerative cartridge 6 through the connecting line 5.

Then, the valve of the cylinder 12 with argon, xenon and krypton is opened, in this case, through the assembly block 13, the regulator 14 and the connecting line 15, an argon-xenon-krypton mixture enters the breathing bag 7, and an oxygen-argon-xenon-krypton mixture with residual nitrogen is formed in it, the composition of which is adjusted according to the readings of the oxygen concentration sensor 17 and the argon-xenon-krypton mixture and the readings of the pressure gauge on the cylinder 12. Then the operating mode of the switchable block of the check valve 3 is set to the connecting line 5 to supply exhaled air towards the regenerative cartridge, if necessary, the valve of the cylinder 9 is closed, the excess of the resulting oxygen-argon-xenon-krypton mixture, with residual nitrogen, is drained through the overpressure valve of the breathing bag 7, and the remaining mixture enters it at inhalation through the regenerative cartridge 6 and the dispenser bag 8 for breathing to the wounded, while, if necessary, using the dispenser bag 8, it is possible to provide him with forced breathing. Further, during exhalation, the mixture passes through the regenerative cartridge 6 into the breathing bag 7, being purified from carbon dioxide and water vapor and enriched with oxygen, and the chemical purification reactions are exothermic, which provides enough heat to heat the mixture in the breathing bag to a temperature of 15-70° C., and then, when inhaled The oxygen-argon-krypton-xenon mixture of gases passes through the regenerative cartridge 6 a second time, once again being purified, heated and enriched with oxygen, thereby closing the breathing cycle.

In the described breathing cycle, argon, krypton and xenon gases are not consumed and do not decrease from the respiratory mixture involved in the respiratory cycle, due to their inertia, but may gradually decrease due to leaks in the mask 1, which can be controlled by a specialized sensor 17, or by analyzing the condition of the wounded taking into account computer readings 16, or based on the experience of the rescuer, and adjusted by adding an argon-krypton-xenon mixture or argon and xenon (krypton) separately from the cylinders 12 into the breathing bag 7. In this case, the dosing of the argon-xenon-krypton mixture from the cylinder 12 through the assembly unit 13, the regulator 14 and the supply line 15 to the breathing bag 7 is carried out the required number of times with control by the pressure gauges of the cylinders 12 and the sensor 17 to achieve a predetermined ratio of oxygen and argon-krypton-xenon for the required period of time up to 6-8 hours.

It is also possible to change the ratios of gases by bleaching part of them through the Mask 1.

In case of difficulty in self-breathing, it is possible to force it with the help of a dispenser bag 8, by periodically pressing on it.

When the resource of the regenerative cartridge 6 is exhausted, it can be replaced with a new one. In addition, it is also possible to replace the cylinder 9 with a helium-oxygen-argon-xenon-krypton medical mixture of gases, cylinders 12 and a chemical heating pad or an electric heating pad battery 11, and the procedure for preparing for work and the algorithm of operation is repeated.

### FIG. 2

FIG. 2 The another version of the device is shown in FIG. 2.

In another variant of the device shown in FIG. 2, instead of a regenerative cartridge 6 and a breathing bag 7, a flexible plastic transparent container 18 with an airtight zipper can be used, in which the patient or injured person is placed entirely and in which a device 19 with a regenerative substance and a chemical carbon dioxide absorber, as well as the Mask 1, which can be worn on the wounded person if necessary, a connecting node 2, the bag—dispenser 8, cylinders 9 and 12 with breathing gas mixtures, regulator 14 for the supply of gas mixture, connecting lines 5 and 15, a wrist laptop computer 16 fixed on the arm of the wounded with a sensor for heart rate (HR), blood oxygen saturation and temperature of the wounded and a sensor 17 for the concentration of oxygen and an argon-krypton mixture-xenon. Gloves can be embedded in container 18 to allow manipulation of the wounded and devices placed in the container (gloves are not shown in the figure). The container 18 must be insulated entirely.

The gas mixture from the cylinder 9 is fed directly into the mask 1, and exhalation is performed into the container 18, and then the excess gas is removed through the overpressure valve 20. The second gas mixture is created directly in the container when the gas mixture is supplied from the cylinder 12. A switchable check valve block 3 is not required in this case. The device 19 with a regenerative substance and a chemical carbon dioxide absorber located in container 18 is activated immediately after placing the injured person in the container.

An example of the use of claimed medicines and devices in real conditions.

In case of an attack of ischemia, injury or injury in the “field” conditions, with acute ischemia, large blood loss and in a state of hypothermia, the following actions are performed:
the wounded person is treated, if possible, with antiseptic and hemostatic agents and a bandage or tourniquet is applied to stop bleeding;
if possible, an injection of an analgesic solution is made by means of using a syringe tube, a tablet preparation of antibiotics is given inside;
fighting asphyxia;
compensate for blood loss, if possible, by infusion of saline solution and other available means;
assemble the claimed device, initiate a chemical or electric heating pad, put on a mask for a sick or injured person and supply a heated mixture of gases according to Option 1—Hearoxxen—krypto gas the required time to recover from hypothermia and stabilize the pre-terminal state;
before the end of the supply of the first respiratory mixture, a regenerative cartridge is initiated and the components forming the second respiratory mixture—argon, xenon and krypton—are added to the gas mixture obtained in the respiratory bag in the required amount to form the “Aroxxen—krypto” gas in an oxygen concentration of 21% by volume up to 60% by volume, xenon from 0.2 to 35% by volume, krypton from 0.2 to 35% by volume, argon the rest in a concentration of at least 25% by volume, and the gas temperature is 15-70° C. due to heating of the gas in the regenerative cartridge, which ensures the transfer of the patient's body to the effective mode providing oxygen to the most important organs, analgesia, psychoemotional stabilization, provides broncholytic and thrombolytic effects, stable pre-terminal condition;

The effect of the application of this invention consists in a significant reduction in the probability of death in ischemic attacks, massive blood loss, acute respiratory failure and combined hypothermia due to an increased likelihood of timely delivery of the patient or wounded to hospital conditions due to withdrawal from hypothermia and a significant extension of the time to maintain the patient or wounded in a viable state in the period of evacuation from field or home conditions.

### DESCRIPTION OF IMPLEMENTATION OPTIONS

Confirmation of the possibility of achieving target effects in the implementation of the invention are the results of special studies carried out under the guidance and with the participation of the authors, which were planned and delivered in a targeted manner to 000 “Research Institute of GEROPRO”, AO “ASM”, Rostov State Medical University, St. Petersburg Institute of Nuclear Physics named after B. P. Konstantinov, Research Establishment “Kurchatov Institute”, Federal State Budgetary Institution “Research Institute of Influenza named after A.A. Smorodintsev” of the Ministry of Health of the Russian Federation, AO “Scientific development and production center “HOUSE OF PHARMACY” with the participation of volunteers and laboratory animals,

The main results of the conducted research were the following facts:

Artificial gas mixtures (AGM) of the claimed formulations, including heated ones, supplied for breathing to laboratory animals (rats, rabbits, pigs), in which acute attacks of cerebral and myocardial ischemia, acute massive blood loss in combination with exogenous hypothermia were simulated, have greater clinical efficacy in terms of prolongation of life and recovery rate The physiological functions of experimental animals are higher than air mixtures with identical (normal or elevated) oxygen content at normal or elevated temperatures.

Artificial gas mixtures of the claimed compositions and physical properties do not have acute and delayed toxic properties and local irritant effect, which has been proven in experiments on various types of laboratory animals (rats, ferrets), including with repeated use of tested AGMs, and with the participation of volunteers.

The ranges of concentrations of the components of the mixture that provide a therapeutic effect were investigated and established, values were found that provide the maximum effect or optimal values of the concentrations of the components, taking into account the error of measurement, creation, dosing of the mixture, for the purpose of maintaining the life of patients with acute cerebral and myocardial ischemia, acute respiratory failure, wounded with massive blood loss and hypothermia, for example:
as a result of the complex of works of the Argon research and development based on the results of research on laboratory animals (State Scientific Center “Institute of Bio-Medical Problems of the Russian Academy of Sciences”) and with the participation of volunteer testers (AO “ASM”), as well as according to foreign sources (Martens A., Ordies S., Vanaudenaerde V. M. et al. A porcine ex vivo lung perfusion model with maximal argon exposure to attenuate ischemia—reperfusion injury//Med. Gas Res.—2017.—Vol. 7.—P. 28-36, etc.) the range of efficiency of a mixture of argon with oxygen for argon with the greatest effect is 30-70% by volume;
as a result of a complex of works at the “Rostov State University Medical University”, taking into account research, as well as literature data, based on the results of studies on laboratory animals and with the participation of volunteer testers, the effective concentrations were estimated helium in a mixture with oxygen, argon, xenon and krypton, with an argon concentration of at least 25% by volume and oxygen from 21% by volume, providing antihypothermic and antihypoxic effects. The helium content should be at least 40% by volume, the temperature of the mixture should be from 30 to 70° C., and the initial temperature of the mixture should preferably be at the lower value of the range of change with an increase to the upper value for about 5 minutes and further maintenance or gradual decrease to 30° C.;
the content of xenon and krypton and their ratios in the mixture are determined by the need to ensure the choice of achieving a hypoalgesic (0.2-10% by volume) or analgesic effect (30-40% by volume) (State Register of Medicines. Xenon (Xenonum). Official publication: in 2 volumes—M.: Medical Council, 2009.—Vol. 2, part 1—568 p.; part 2—560 p.);
the need to use krypton is due to its properties (Kussmaul A. R., “The biological effect of krypton on animals and humans under high pressure conditions”, dissertation for the degree of Candidate of Biological Sciences, “Institute of Bio-Medical Problems of the Russian Academy of Sciences”, Moscow, 2007.), as well as the following:
the critical temperature of krypton is 209.35 K, which allows it to be used in temperate latitudes in winter in the field and at Arctic temperatures without the risk of condensation (xenon—289.74 K); krypton, having a xenon-like effect, is 5-8 times cheaper.
a large permissible total relative error in setting the parameters of the mixture, reaching 10%, is due to the need to simplify the design and operation in the field and, at the same time, in the effective ranges found, does not significantly affect the effect of using the claimed drugs at concentrations of components in the claimed ranges;
due to the wide ranges of effective concentrations of the components of the mixture and the smooth change in the effects of their effects, it is possible to create a family of drugs and a family of devices to implement the method within the declared concentration ranges, or (and) instructions for doctors to regulate the composition of the supplied mixture (qualitative and quantitative), within the general framework of the proposed device
   For example:
the device for regions with hot and warm climates can be lightweight, implemented according to the scheme FIG. 1, with a reduced capacity of the cylinder with mixture 1, the absence of insulation, the use of xenon mixtures in full, if necessary, up to 35% by volume;
   the device for regions with moderate climate:
in summer—a lightweight version, implemented according to the scheme FIG. 1, with a reduced capacity of the cylinder with mixture 1, the absence of insulation, the use of xenon and krypton mixtures in full;
in winter—the full version according to the scheme FIG. 2, with an increased capacity of the cylinder with mixture 1, insulation, use in mixtures of xenon and krypton in full or with a predominance of krypton;
the device for regions with an Arctic climate is a complete version according to the scheme FIG. 2, with an increased set of chemical heaters and minimization of xenon in mixtures 1 and 2.

### EXAMPLES

Modeling of the pathological process (acute ischemia, massive blood loss in combination with exogenous hypothermia) was carried out on 2 types of laboratory animals (rats and rabbits) after preliminary anesthesia. Acute myocardial ischemia in rats was modeled by ligation of coronary vessels. Acute cerebral ischemia in rats was modeled by focal ischemia caused by occlusion of the middle cerebral artery (OMCA). Acute cerebral ischemia in rabbits was modeled by ligation of both carotid arteries and subsequent blood sampling from one of the carotid arteries above the ligature in a volume of 40% of the circulating blood volume (approximately 4% of the rabbit's body weight). Massive acute blood loss in rats up to 50% of the volume of circulating blood was modeled by transcutaneous puncture of the heart, in rabbits—40-45% of the volume of circulating blood—by puncture of the femoral artery from a soft tissue incision. Exogenous hypothermia in both cases was modeled by pouring cold water on the operated animal, drying it with a towel and then placing it in a cold chamber with a temperature of minus 30° C. for 10 minutes, preventing frostbite and freezing of the animal. Body temperature was controlled rectally.

After modeling, the animals were randomly sorted into a control or experimental group.

The individuals of the control group were placed in a chamber with air temperature of 20+2° C., and oxygen content of 23+2% by volume, 40+2% by volume and 58+2% by volume.
1 The individuals of the experimental group were first placed under a “hood”, under which a heated mixture of “Hearoxxen”—krypto” gases with a helium content of 45-50% by volume for 5 minutes and 20 minutes, and further, the animals were moved to a chamber with a mixture of gases “Aroxxen”—krypto” with an initial temperature from 40+2° C. to 60+2° C., and an oxygen content of 23+2% by volume, 40+2% by volume and 58+2% by volume, argon content 33+2% by volume, krypton—2+1% by volume, xenon—2+1% by volume and the rest is nitrogen.

The general condition, rectal temperature, and death of animals were observed and recorded. Remote monitoring (mortality assessment) of the animals continued for several days.

The survival and recovery of vital functions of rats (21 individuals in the experimental group) or rabbits (9 individuals in each group) were observed after modeling ischemia or acute massive blood loss on the background of hypothermia, evaluated in paired experimental and control groups.

As a result of experiments according to claim 1, it was obtained:
in experiments on ischemia in rabbits, the survival rate during exposure to the gas of the claimed 2 composition for up to 4 hours was 100%, in the control group—33%. After cessation of exposure to medicinal gas of the claimed composition 2, the survival rate in the experimental group was 77.8%.
in experiments with modeling massive blood loss, survival in experimental groups of laboratory animals was significantly higher: rats—18 out of 21 (87.5%), rabbits—9 out of 9 (100%) than in control: rats—5 out of 12 (58%), rabbits—3 out of 8 (37.5%);
the oxygen content in the gas mixture in the experimental groups did not significantly affect the survival of laboratory animals, unlike the control group, where 6 animals died at 23+2% by volume, 4 animals at 40+2% by volume and 3 animals at 58+2% by volume.
2 The individuals of the experimental group of rats were first placed under a “hood”, under which a heated mixture of gases was supplied according to Variant 1 of the invention with a helium content of 45-50% by volume for 5 minutes, and further, the animals were moved into a chamber with a mixture of gases according to Variant 2 of the invention with an initial temperature from 40+2° C. to 60+2° C., and an oxygen content of 40+2% by volume, krypton—10+2% by volume, xenon—2+1% by volume and argon—the rest (about 43-53% by volume).

Survival and recovery of vital functions of rats after acute massive blood loss on the background of hypothermia were observed.
3 The individuals of the experimental group of rats were first placed under a “hood”, under which a heated mixture of gases was supplied according to Variant 1 of the invention with a helium content of 45-50% by volume for 5 minutes, and further, the animals were moved into a chamber with a mixture of gases according to Variant 2 of the invention with an initial temperature from 40+2° C. to 60+2° C., and an oxygen content of 40+2% by volume, krypton—18+2% by volume, xenon—2+1% by volume and argon—the rest (about 35-45% by volume).

As a result of experiments according to paragraphs 2 and 3, the following was obtained:
survival rates in groups of rats given a mixture containing 10+2% by volume of krypton and 18+2% by volume of krypton are the same.
the recovery of functions in the experimental group of rats at a concentration of krypton in a mixture of 18+2% by volume occurs somewhat faster (about 1.2-1.3 times) than in the group of rats at a concentration of krypton in a mixture of 10+2% by volume.
4 The individuals of the experimental group of rats were first placed under a “hood”, under which a heated mixture of gases was supplied according to claim 1 of the claims with a helium content of 45-50% by volume for 10 minutes, and further, the animals were moved into a chamber with a mixture of gases according to claim 2 of the claims with an initial temperature from 20+2° C. to 40+2° C., and an oxygen content of 40+2% by volume, krypton—2+1% by volume, xenon—2+1% by volume and the rest is argon.

Survival and recovery of vital functions of rats after acute massive blood loss on the background of hypothermia were observed.
5 The individuals of the experimental group of rats were first placed under a “hood”, under which a heated mixture of gases was supplied according to claim 1 of the claims with a helium content of 45-50% by volume for 20 minutes, and further, the animals were moved into a chamber with a mixture of gases according to claim 2 of the claims with an initial temperature from 20+2° C. to 40+2° C., and an oxygen content of 40+2% by volume, krypton—2+1% by volume, xenon—2+1% by volume and the rest is argon.

Survival and recovery of vital functions of rats after acute massive blood loss on the background of hypothermia were observed.

As a result of experiments according to paragraphs 4 and 5, it was obtained:
survival rates in the group of rats who were given 1 mixture for 10 minutes are better than survival rates in the group of rats who were given 1 mixture for 20 minutes, which may indicate that there is an optimum efficiency of procedures when applying a warming mixture and switching to a maintenance mixture 2.

### INDUSTRIAL APPLICABILITY

The analysis of the result of numerous experimental studies involving animals and volunteers, including the examples given, allowed us to determine the optimal effective composition for the variants of the gases “Hearoxxen—krypto” and “Aroxxen—krypto” to maintain human life with severe blood loss, acute cerebral and myocardial ischemia, acute respiratory failure, and with hypothermia.

The general composition of the medical gases “Hearoxxen—krypto” and “Aroxxen—krypto” and examples of target variants of the compositions of the claimed medicinal gases due to the symptoms of the patient's condition and ambient temperature are shown in Table 1.

The pharmacokinetics and pharmacodynamics of the claimed medicinal medical gases, their breadth of action, make it possible to produce a wide range of drugs with targeted action and applicability parameters within the claimed ranges of effective concentrations or create effective gas mixtures required for the current condition of a particular patient within the claimed compositions on site using the claimed devices.

Table 1—Examples of formulations of variants of the claimed medicinal products of mixtures of medical gases for maintaining human life with severe blood loss, acute cerebral and myocardial ischemia, acute respiratory failure, and hypothermia.

TABLE 1
Examples of formulations of variants of the claimed medicinal products of mixtures of medical gases for maintaining the life of a
person with severe blood loss, acute cerebral and myocardial ischemia, acute respiratory failure, and hypothermia

| | Con- | | The quantitative composition of | | | | | | | | | |
|---|---|---|---|---|---|---|---|---|---|---|---|---|
| | ditional | | the medicine, % by volume. | | | | | | | | | |
| | medicinal | | | | | | | impurities, | | | | |
| | product | Ex- | | | | | | amount, | T, | | | |
| No. | name | amples | He | O₂ | Xe | Kr | Ar | ppm | ° C. | Purpose | Effect | Conditions of use |
| 1 | Hearoxxen- | | 40-50 | no | 0.2-5 | 2-5 | the | <500 | 30-70 | maintaining | warming, | storage at a |
| | krypto | | (45 ± | more | | | rest | | | human life at | antihypoxic, | temperature above |
| | | | 5) | than 21 | | | ≥25 | | | the initial stage | analgesic, | 16° C., application |
| | | | | | | | | | | of withdrawal | bronchodilator, | for a short period |
| | | | | | | | | | | from | | of 5-30 minutes |
| | | | | | | | | | | hypothermia, | | |
| | | | | | | | | | | relief of | | |
| | | | | | | | | | | hypoxia, with | | |
| | | | | | | | | | | large blood | | |
| | | | | | | | | | | loss, acute | | |
| | | | | | | | | | | cerebral | | |
| | | | | | | | | | | myocardial | | |
| | | | | | | | | | | ischemia, | | |
| | | | | | | | | | | acute | | |
| | | | | | | | | | | respiratory | | |
| | | | | | | | | | | failure, with | | |
| | | | | | | | | | | hypothermia. | | |
| | | Ex- | 45 | 23 | 2 | 5 | the | [symbol] | [symbol] | | warning, | storage at a |
| | | ample | | | | | rest | | | | antihypoxic | temperature above |
| | | 1.1 | | | | | 25 | | | | combined | 16° C., application |
| | | | | | | | | | | | analgesic, | for a short period |
| | | | | | | | | | | | bronchodilator, | of 5-30 minutes |
| | | | | | | | | | | | decrease in | |
| | | | | | | | | | | | blood viscosity | |
| | | Ex- | 40 | 23 | 2 | 5 | the | [symbol] | [symbol] | | warning, | storage at a |
| | | ample | | | | | rest | | | | antihypoxic | temperature above |
| | | 1.2 | | | | | 30 | | | | enhanced, | 16° C., application |
| | | | | | | | | | | | combined | for a short period |
| | | | | | | | | | | | analgesic, | of 5-30 minutes |
| | | | | | | | | | | | bronchodilator, | |
| | | | | | | | | | | | decrease in | |
| | | | | | | | | | | | blood viscosity | |
| | | Ex- | 40 | 25 | 5 | 5 | the | [symbol] | [symbol] | | warning, | storage at a |
| | | ample | | | | | rest | | | | antihypoxic | temperature above |
| | | 1.3 | | | | | 25 | | | | enhanced, | 16° C., application |
| | | | | | | | | | | | analgesic | for a short period |
| | | | | | | | | | | | enhanced, | of 5-30 minutes |
| | | | | | | | | | | | bronchodilator, | |
| | | | | | | | | | | | decrease in | |
| | | | | | | | | | | | blood viscosity | |
| | | Ex- | 50 | 22.8 | 0.2 | 2 | the | [symbol] | [symbol] | | warming | storage and |
| | | ample | | | | | rest | | | | enhanced, | application at |
| | | 1.4 | | | | | 25 | | | | antihypoxic | ambient |
| | | | | | | | | | | | analgesic, | temperatures from |
| | | | | | | | | | | | bronchodilator, | minus 50° C. to |
| | | | | | | | | | | | decrease in | plus 40° C, 5-30 |
| | | | | | | | | | | | blood viscosity. | minutes |
| 2 | Aroxxen- | | 0 | 21-60 | 0.2-35 | 0.2-35 | the | <500 | 15-70 | maintaining | toxic analgesic, | universal, storage |
| | krypto | | | | | | rest | | | human life at | cybernetic, | at temperatures |
| | | | | | | | 25 | | | the stage of | broncholytic | above 16° C., |
| | | | | | | | | | | arresting an | anxiolytic | long-term |
| | | | | | | | | | | attack before | | application |
| | | | | | | | | | | delivery to | | |
| | | | | | | | | | | the hospital | | |
| | | Ex- | 0 | 60 | 2 | 3 | the | [symbol] | [symbol] | | antihypoxic | universal, storage |
| | | ample | | | | | rest | | | | analgesic, | and application at |
| | | 2.1 | | | | | 35 | | | | bronchodilator, | the temperature |
| | | | | | | | | | | | for adults, | above 16° C. |
| | | Ex- | 0 | 40 | 2 | 28 | the | [symbol] | [symbol] | | for adults with | universal, storage |
| | | ample | | | | | rest | | | | severe injuries | and application at |
| | | 2.2 | | | | | 30 | | | | and seizures, | the temperature |
| | | | | | | | | | | | antihypoxic | above 16° C. is |
| | | | | | | | | | | | enhanced, | recommended |
| | | | | | | | | | | | analgesic | |
| | | | | | | | | | | | enhanced, | |
| | | | | | | | | | | | bronchodilator, | |
| | | | | | | | | | | | anxiolytic, | |
| | | | | | | | | | | | anticoagulant. | |
| | | Ex- | 0 | 40 | 0.2 | 29.8 | the | [symbol] | [symbol] | | for adults with | universal, storage |
| | | ample | | | | | rest | | | | severe injuries | and application at |
| | | 2.3 | | | | | 30 | | | | and seizures, | the ambient |
| | | | | | | | | | | | antihypoxic | temperature from |
| | | | | | | | | | | | enhanced, | minus 50° C. to |
| | | | | | | | | | | | analgesic | plus 40° C. |
| | | | | | | | | | | | enhanced, | |
| | | | | | | | | | | | broncholytic, | |
| | | | | | | | | | | | anxiolytic, | |
| | | | | | | | | | | | attention- | |
| | | | | | | | | | | | anticoagulant [symbol] | |
| | | Ex- | 0 | 30 | 35 | 5 | the | [symbol] | [symbol] | | for adults in a | storage and |
| | | ample | | | | | rest | | | | terminal state, | application at the |
| | | 2.4 | | | | | 30 | | | | antihypoxic | temperature above |
| | | | | | | | | | | | enhanced, | 16° C. |
| | | | | | | | | | | | analgesic | |
| | | | | | | | | | | | enhanced, | |
| | | | | | | | | | | | cybernetic, | |
| | | | | | | | | | | | narcotic, | |
| | | | | | | | | | | | broncholytic, | |
| | | | | | | | | | | | anxiolytic. | |
| | | Ex- | 0 | 30 | 0.2 | 5 | the | [symbol] | 20-40 | | for new-borns | universal, storage |
| | | ample | | | | | rest | | | | | and application at |
| | | 2.5 | | | | | >25 | | | | | the temperature |
| | | | | | | | 25-64, | | | | | above 16° C. is |
| | | | | | | | 8 | | | | | recoommended |
| | | | | | | | (+N₂) | | | | | |
| | | Ex- | 0 | 21 | 9 | 35 | the | [symbol] | 15-70 | | for adults in a | storage and |
| | | ample | | | | | rest | | | | state of pain | application at the |
| | | 2.6 | | | | | 35 | | | | shock, | temperature above |
| | | | | | | | | | | | antihypoxic, | 16° C. |
| | | | | | | | | | | | analgesic | |
| | | | | | | | | | | | enhanced, | |
| | | | | | | | | | | | cybernetic, | |
| | | | | | | | | | | | broncholytic, | |
| | | | | | | | | | | | anxiolytie, | |
| | | | | | | | | | | | anticoagulant. | |
| | | Ex- | 0 | 21 | 1 | 10 | the | [symbol] | — | | for the stage of | universal, storage |
| | | ample | | | | | rest | | | | seizure relief, | and application at |
| | | 2.7 | | | | | 68 | | | | preconditioning, | the ambient |
| | | | | | | | | | | | rehabilitation, | temperature from |
| | | | | | | | | | | | prevention, | minus 50° C. to |
| | | | | | | | | | | | antihypoxic, | plus 40° C. |
| | | | | | | | | | | | analgesic, | |
| | | | | | | | | | | | broncholytic, | |
| | | | | | | | | | | | anxiolytic. | |
| | | | | | | | | | | | anticoagulant | |

[symbol]

indicates data missing or illegible when filed

1. A medicinal product for maintaining the life of a person with severe blood loss, acute cerebral and myocardial ischemia, acute respiratory failure, and hypothermia, is the product comprising
a mixture of gases comprising oxygen, xenon and argon, wherein the mixture of gases contains helium in the concentration range from 40 to 50% by volume, argon in the concentration range from 25 to 35% by volume, xenon in the concentration range from 0.2 to 5% by volume, krypton in the concentration range from 2 to 10% by volume and oxygen—at least 21% by volume, up to 100% by volume in total, the temperature of the gas mixture is 30-70° C.

2. A medicinal product for maintaining the life of a person with severe blood loss, acute cerebral and myocardial ischemia, acute respiratory failure, and hypothermia, is the product comprising:
a mixture of gases comprising oxygen, xenon, argon, wherein the mixture of gases contains oxygen in the concentration range from 21% by volume to 60% by volume, xenon in the concentration range from 0.2 to 35% by volume, krypton in the concentration range from 0.2 to 35% by volume, argon the rest in a concentration of at least 25% by volume, and the gas temperature is 15-70° C.

3. A device for maintaining the life of a person with severe blood loss, acute cerebral and myocardial ischemia, acute respiratory failure, and hypothermia with the help of medicines according to claim 1 (in the form of respiratory mixtures of medical gases), characterized by the presence of a facial part—mask 1 with a mouthpiece located in it, a connective node 2, breathing bag 7 with overpressure valve, a bag—a dispenser 8 connected to a switchable check valve block and a regeneration cartridge 6 connected to a connecting line with a breathing bag 7, a cylinder with argon-xenon-krypton with a mixture of gases 12 or two smaller cylinders with argon and xenon and krypton in the assembly and with a three-way valve for alternately supplying gases to the breathing bag 7 through the gas mixture supply regulator 14, through connecting lines 15 for supplying the mixture to the breathing bag 7, a wrist laptop computer 16 with a heart rate sensor and oxygen saturation blood, oxygen and xenon concentration sensor in the breathing bag 7, designed to monitor and control the condition of the wounded, which is also characterized by, that it is additionally equipped with a balloon 9 with a medicinal product—helium—an oxygen-argon-xenon-krypton mixture of gases, with a valve and a pressure gauge mounted on it, connected via a connecting line 10 to the mask with the possibility of supplying a mixture of gases directly into the mask 1, a chemical or electric heating pad 11c with a heat exchange connecting line, a switchable check valve block 3 installed between the outlet of the connecting node of the mask and the regeneration cartridge 6 pneumatically connected to a dispenser bag 8 and an exhalation line to the outside, an oxygen concentration sensor and an argon-krypton-xenon mixture.

4. The device according to claim 3, characterized in that the mask, the connecting node, the switchable block of the check valve, a chemical heating pad with a heat exchange connecting line, a breathing bag, connecting lines through which a breathing mixture of gases is supplied to it, a metering bag with a check valve are provided with thermal insulation.

5. The device according to claim 3, characterized in that, for long-term maintenance of human life, it additionally includes a transparent plastic container with an airtight zipper and built-in gloves for placing the injured person in it entirely, a mask placed in it connected to a dispenser bag with the possibility of putting it on the wounded person to stimulate breathing, and a device with a regenerating substance, as well as a dispenser bag with switchable check valve and connecting line, a cylinder with a mixture of gases or two separate cylinders of a smaller capacity with argon and xenon in the assembly and with a three-way valve for alternating gas supply, a gas mixture supply regulator, a connecting line for feeding the mixture into a dispenser bag, while the device is additionally equipped with a cylinder with a helium-oxygen-argon-xenon-krypton mixture of gases installed on it is equipped with a valve and a pressure gauge connected through a heat exchange connecting line to the mask with the possibility of supplying a mixture of gases directly into the mask, a chemical heating pad with a heat exchange connecting line and an overpressure valve hermetically installed in the wall of a plastic container, as well as in a cylinder 12 with a mixture of argon-xenon-krypton gases.

6. The device according to claim 5, characterized in that a transparent plastic container is provided with thermal insulation entirely.
